(19) United States
(12) Patent Application Publication
(10) Pub. No.: US 2020/0196869 A1
(43) Pub. Date: Jun. 25, 2020

(54) A HAND-HELD BIOPHOTONIC MEDICAL DEVICE, METHOD AND SYSTEM FOR MULTIMODAL AND MULTISPECTRAL IMAGING OF A TISSUE

(71) Applicant: SUBHASH NARAYANAN, Trivandrum (IN)

(72) Inventor: SUBHASH NARAYANAN, Trivandrum (IN)

(21) Appl. No.: 16/348,509

(22) PCT Filed: May 21, 2018
(86) PCT No.: PCT/IB2018/053560

§ 371 Date: May 9, 2019

(30) Foreign Application Priority Data
May 19, 2017 (IN) 201741017679

(51) Int. Cl. A61B 5/00; A61B 5/1455

(52) U.S. Cl. CPC A61B 5/0037; A61B 5/0071; A61B 2576/00; A61B 5/14551; A61B 5/7246

(57) ABSTRACT

The invention relates to a Handheld Biophotonic Medical (HBM) device for multimodal and multispectral imaging of a tissue. The HBM device comprises a hardware switch that provides trigger pulses to control unit of the HBM device, which controls an illumination unit to illuminate the tissue. Further, HBM device controls a miniature monochrome imaging device to stream live video image of tissue fluorescence and to capture images of tissue fluorescence and diffusely reflected light in real-time based on the light of specific wavelengths received from a collection optics unit upon illumination of the tissue. The control unit transmits the captured images to a computing device that determines grade of cancer and inflammation by analysing the captured images. The HBM device is light weighted, portable, can be inserted into body parts such as oral cavity, cervix and can also be mounted on endoscopes to examine internal organs of body.

### FIELD OF THE INVENTION

The present subject matter relates generally to a medical device, and more particularly, but not exclusively to a hand-held biophotonic medical device, a method and a system for multimodal and multispectral imaging of a tissue.

### BACKGROUND OF THE INVENTION

Nowadays, cancer is a growing concern across the world. Burden of cancer is alarmingly high and it is expected to grow from 10 million new cases globally in the year 2000 to 15 million new cases globally in the year 2020. Many types of cancer grow from epithelial tissues covering inner and outer linings of a human body, such as gastrointestinal (GI) tract, oral cavity, cervix, colon and stomach. The Oropharyngeal cancer type is a significant component of the global cancer burden and is a sixth most common type of cancer internationally. Early detection of localized lesions and pre-malignant to dysplastic changes in the oral cavity facilitates adoption of appropriate preventive and treatment strategies that can influence disease outcomes and reduce mortality. Early detection of various changes in oral mucosa leading to cancer can save lives of the people suffering from cancer.

However, in normal clinical settings, it is extremely challenging for the clinicians to visually identify the most malignant site in a lesion for tissue biopsy and pathology. Therefore, the patients may have to undergo multiple biopsies that are painful to achieve appropriate diagnosis. Existing techniques for screening patients for oral cancer and precancerous lesions include obtaining a fluorescence spectra and diffuse reflectance spectra that are analysed using multivariate analytical techniques to detect cancer. As an example, a Multispectral optical imaging Digital Microscope (MDM) is a device that acquires in-vivo images of oral tissue fluorescence, along with recording of narrow band (NB) reflectance and orthogonal polarized reflectance to improve accuracy in detection of cancer. Though the MDM improves accuracy in detecting cancer, there still exists discrepancy as the device cannot be inserted into the human body. Therefore, the in-vivo images are obtained by fixing cameras outside the human body which cannot be completely relied upon as the fixed position of the cameras may not capture a clear image of the affected regions inside the human body. Also, these types of devices for detecting cancer are bulky and heavy in nature, thereby lacking a portability factor and also ease of handling the device. Further, these types of devices may achieve selected collection of the diffusely reflected light and tissue fluorescence by using filters such as Liquid Crystal Tunable Filters that are extremely expensive, thereby increasing cost of the device on the whole. Also, most of the existing techniques use either fluorescence imaging or diffuse reflectance imaging for detecting abnormalities in tissue or a combination of fluorescence and diffuse reflectance imaging. However, there exists no device that could perform multimodal imaging combining tissue autofluorescence, absorption and diffuse reflectance.

US2012078524 discloses a system and method for determining a diagnosis of a test biological sample. A system comprising a first illumination source to illuminate a sample, a first detector for generating a fluorescence data set of said sample, a means for determining a region of interest, a second illumination source to illuminate said region of interest, a second detector to generate a Raman data set of said region of interest, and a means for determining a diagnosis of said sample. A method comprising illuminating a sample, generating a fluorescence data set of said sample, and assessing the fluorescence data set to identify a region of interest, illuminating a region of interest, and generating Raman data set. This Raman data set may be assessed to determine a diagnosis of the sample. A diagnosis may include a metabolic state, a clinical outcome, a disease progression, a disease state, and combinations thereof. The main drawback of this invention is that a plurality of spectral information processing devices such as a tunable fluorescence filter, tunable Raman filter, dispersive spectrometer, plurality of detectors, a fiber array spectral translator, variety of filters and a polarized beam splitter substantially make this system bulky and expensive. Further, this invention is difficult in integrating to a handheld device.

WO2014118326 discloses a system and method for characterization and/or calibration of performance of a multispectral imaging (MSI) system equipping the MSI system for use with a multitude of different fluorescent specimens while being independent on optical characteristics of a specified specimen and providing an integrated system level test for the MSI system. A system and method are adapted to additionally evaluate and express operational parameters performance of the MSI system in terms of standardized units and/or to determine the acceptable detection range of the MSI system. This invention does not disclose multimodal imaging of the tissue such as fluorescence, absorption and diffuse reflectance. This invention is not handheld, lightweight and portable which can be used for in vivo application for diagnosis.

Therefore, there exists a need of cost effective and easy to use technology that is handheld, lightweight, portable and can perform multimodal imaging of tissues such as tissue fluorescence, absorption, scattering, thermal imaging and diffuse reflectance etc. Further, there is need of technology that can perform in vivo diagnosis and determine grade of cancer or inflammation in the tissue accurately.

### SUMMARY OF THE INVENTION

One or more shortcomings of the prior art may be overcome and additional advantages may be provided through the present disclosure. Additional features and advantages may be realized through the techniques of the present disclosure. Other embodiments and aspects of the disclosure are described in detail herein and are considered a part of the claimed disclosure.

Disclosed herein is a Hand-held Biophotonic Medical (HBM) device for multimodal and multispectral imaging of a tissue. The HBM device comprises an illumination unit comprising a predefined combination of one or more illuminating devices emitting at one or more predefined wavelengths with predefined bandwidths to illuminate the tissue through a polarizer. The HBM device further comprises a miniature monochrome imaging device configured to stream live video of tissue fluorescence upon absorption of incident light by constituents of the tissue. The miniature monochrome imaging device captures one or more images of the tissue fluorescence, upon the absorption of the incident light by the constituents of the tissue, and diffusely reflected light due to multiple elastic scattering of the incident light in the tissue, in real-time. Further, the HBM device comprises a hardware switch configured to provide one or more trigger pulses to a control unit of the HBM device when triggered. Furthermore, the HBM comprises a collection optics unit comprising a lens that collects the tissue fluorescence and the diffusely reflected light from the tissue upon illumination and directs it through a crossed polarizer (105b) to a tailored optical filter. The tailored optical filter transmits light in a predefined wavelength range covering the tissue fluorescence and the diffusely reflected light. The control unit receives the one or more trigger pulses from the hardware switch. Further, the control unit drives the one or more illuminating devices sequentially to illuminate the tissue for a particular duration upon receiving the one or more trigger pulses. Furthermore, the control unit controls the miniature monochrome imaging device upon receiving the one or more trigger signals to capture the one or more images. Finally, the control unit transmits the one or more images to the computing device for display and further processing.

Further, the present disclosure relates to a system for multimodal and multispectral imaging of a tissue. The system comprises a Hand-held Biophotonic Medical (HBM) device and a computing device. The HBM device comprises an illumination unit consisting of a predefined combination of one or more illuminating devices emitting at one or more predefined wavelengths with predefined bandwidths to illuminate the tissue through a polarizer. The HBM device further comprises a miniature monochrome imaging device configured to stream live video of tissue fluorescence upon absorption of incident light by constituents of the tissue. The miniature monochrome imaging device captures one or more images of the tissue fluorescence upon the absorption of the incident light by the constituents of the tissue and diffusely reflected light due to multiple elastic scattering of the incident light in the tissue, in real-time. Further, the HBM device comprises a hardware switch configured to provide one or more trigger pulses to a control unit of the HBM device. Furthermore, the HBM device comprises a collection optics unit comprising a lens that collects the tissue fluorescence and the diffusely reflected light from the tissue upon illumination, and directs it through a crossed polarizer (105b) to a tailored optical filter. The tailored optical filter transmits light in a predefined wavelength range covering the tissue fluorescence and the diffusely reflected light. The control unit receives the one or more trigger pulses from the hardware switch. Further, the control unit drives the one or more illuminating devices sequentially to illuminate the tissue for a particular duration upon receiving the one or more trigger pulses. Upon illuminating the tissue, the control unit controls the miniature monochrome imaging device upon receiving the one or more trigger signals to capture the one or more images. Finally, the control unit is configured to transmit the one or more images captured to the computing device for display and further processing. The one or more illuminating devices and the miniature monochrome imaging device can be triggered sequentially via the hardware switch. Further, the computing device receives at least one of the live video of the tissue fluorescence and the one or more images of the tissue fluorescence and the diffusely reflected light of the tissue captured by the miniature monochrome imaging device upon illumination of the tissue by the one or more illuminating devices of the HBM device. Further, the computing device detects changes in intensity of oxygenated haemoglobin absorption in tissue, at predefined wavelength range in the tissue by analysing the one or more images. Further, the computing device obtains one or more pseudo coloured images by false colouring the one or more images captured by the miniature monochrome imaging device. Furthermore, the computing device determines image intensity ratio values of the one or more images captured by the miniature monochrome image capturing device in the predefined wavelength range. Upon determining the image intensity ratio values, the computing device identifies Regions of Interest (ROI) comprising a maximum change in the image intensity ratio values when compared to predefined standard ratio values, wherein the predefined standard ratio values are related to the ROI of a similar (corresponding) site in a normal (healthy) tissue. Finally, the computing device determines at least one of a grade of cancer or a grade of inflammation in the tissue automatically based on the intensity of the oxygenated haemoglobin absorption and by correlating the image intensity ratio values obtained from the one or more images using a diagnosing algorithm.

Furthermore, the present disclosure comprises a method for multimodal and multispectral imaging of a tissue. The method comprises streaming, by a Hand-held Biophotonic Medical (HBM) device, a live video of tissue fluorescence upon powering on the HBM device. The live video is obtained using a miniature monochrome imaging device associated with the HBM device. Further, the method comprises receiving, by a Hand-held Biophotonic Medical (HBM) device, one or more trigger pulses from a hardware switch of the HBM device. Upon receiving the one or more trigger pulses, the HBM device triggers one or more illuminating devices sequentially to illuminate the tissue. Further, the HBM device controls a miniature monochrome imaging device to capture one or more images of the tissue fluorescence upon absorption of incident light by constituents of the tissue and diffusely reflected light due to multiple elastic scattering of the incident light at a predefined wavelength range from the tissue in real-time using the miniature monochrome imaging device and a collection optics unit associated with the HBM device. Finally, the HBM device transmits the one or more images to the computing device for display.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE ACCOMPANYING DRAWINGS

The accompanying drawings, which are incorporated in and constitute a part of this disclosure, illustrate exemplary embodiments and, together with the description, serve to explain the disclosed principles. In the figures, the left-most digit(s) of a reference number identifies the figure in which the reference number first appears. The same numbers are used throughout the figures to reference like features and components. Some embodiments of system and/or methods in accordance with embodiments of the present subject matter are now described, by way of example only, and with reference to the accompanying figures, in which:

FIG. 1A shows an exemplary system illustrating process for multimodal and multispectral imaging of a tissue in accordance with some embodiments of the present disclosure;

FIG. 1B and FIG. 1C show a top view and a side view of the Hand-held Biophotonic Medical (HBM) device respectively in accordance with some embodiments of the present disclosure;

FIG. 1D shows an exemplary graph illustrating transmission characteristics of a tailored optical filter in accordance with some embodiments of the present disclosure;

FIG. 1E shows internal architecture of the system for multimodal and multispectral imaging of a tissue in accordance with some embodiments of the present disclosure; and

FIG. 1F shows an exemplary application layer of miniature monochrome imaging device and a computing device in accordance with some embodiments of the present disclosure.

FIG. 2 shows a flowchart illustrating a method for multimodal and multispectral imaging of a tissue in accordance with some embodiments of the present disclosure.

FIG. 2a elucidates a flow diagram for the image acquisition and image processing of tissues in accordance with an embodiment in the present invention;

FIG. 3 shows a set of images captured by the handheld device to show malignant site for biopsy in accordance with an embodiment in the present invention;

FIG. 4 shows various anatomical sites of the oral cavity to be diagnosed in accordance with an embodiment in the present invention;

FIG. 5 shows a scatter plot diagram correlating the image ratio value (R610/R545) obtained for patients with leukoplakia compared with that of healthy subjects in accordance with the embodiment in the present invention; and

FIG. 6 elucidates the topological structure of handheld device 101 with the computing unit 113.

It should be appreciated by those skilled in the art that any block diagrams herein represent conceptual views of illustrative systems embodying the principles of the present subject matter. Similarly, it will be appreciated that any flow charts, flow diagrams, state transition diagrams, pseudo code, and the like represent various processes which may be substantially represented in computer readable medium and executed by a computer or processor, whether or not such computer or processor is explicitly shown.

### DETAILED DESCRIPTION OF THE INVENTION

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the forms disclosed, but on the contrary, the disclosure is to cover all modifications, equivalents, and alternative falling within the scope of the disclosure.

The terms “comprises”, “comprising”, “includes” or any other variations thereof, are intended to cover a non-exclusive inclusion, such that a setup, device or method that includes a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or method.

The present disclosure provides a Hand-held Biophotonic Medical (HBM) device for multimodal and multispectral imaging of a tissue. The multiple modes included in this disclosure are fluorescence, absorption, transmittance, reflectance, diffuse reflectance, elastic scattering, inelastic scattering (Raman spectroscopy), photoacoustic and thermal imaging. In some embodiments, the fluorescence may be at least one of autofluorescence or photosensitizer-induced fluorescence. The HBM device is a light weighted, easily handled, portable device, and can be easily inserted into parts of a body such as oral cavity, cervix and the like. In some embodiments, the HBM device may be fixed to an external body and used as a fixed device instead of a hand-held device. Further, the HBM device can be adapted for coupling to endoscopes to examine internal organs of the body. The HBM device comprises a hardware switch that provides one or more trigger pulses to a control unit of the HBM device when triggered. In some embodiments, the one or more trigger pulses may be provided using a computing device connected with the HBM device. Upon receiving the trigger pulse, the control unit activates the illumination unit that in turn sequentially triggers one or more illuminating devices present in the illumination unit. The present disclosure discloses use of multiple Light Emitting Diodes (LEDs) of one or more predefined wavelengths for illuminating the tissue. Optical narrowband interference filters are alternatively mounted on top of the LEDs to reduce the spectral emission bandwidth wherever required. The use of LEDs instead of other light sources such as white light source, tungsten halogen lamp, mercury-xenon lamp and the like eliminates the need for expensive filters such as liquid crystal tunable filters, acousto-optic tunable filters and the like in the detection path for multispectral imaging. Further, the HBM device comprises a miniature monochrome imaging device configured to stream live video of tissue fluorescence upon absorption of incident light by constituents of the tissue and capture one or more images of the tissue fluorescence and diffusely reflected light in the tissue in real-time upon illumination of the tissue with polarised light of predefined wavelength and predefined bandwidth. The one or more images captured by the miniature monochrome imaging device is representative of biochemical, morphological and structural changes in tissue during malignant transformation and is based on the absorption, elastic scattering and fluorescence of light in a predefined wavelength and spatial range received by the collection optics unit. The collection optics unit may include, but not limited to, a lens, a crossed polarizer and a tailored optical filter. The wide angle lens collects the tissue fluorescence and the diffusely reflected light and directs it to a monochrome sensor via a tailored optical filter and a crossed polarizer that minimizes/removes the specular reflection component in the diffusely reflected light. The tailored optical filter is an interference band filter that transmits only light of one or more predefined wavelengths covering tissue fluorescence, and the diffusely reflected light at the HbO2 absorption wavelengths of 545 and 575 nm, and at the HbO2 absorption-free wavelength around 610 nm. In the present embodiment for oral cancer screening, the tailored optical filter transmits light in the 450-620 nm wavelength range. Whereas for cervical cancer screening, the tailored optical filter to block the 365 nm LED light used for inducing collagen fluorescence, while transmitting the collagen fluorescence and the elastically scattered light from the other 3 LEDs used for diffuse reflectance imaging. Further, the interference filters for spectral narrowing of LED light has a bandwidth (FWHM) of 8±2 nm, centered at 546±2 nm, 578±2 nm, and 610±2 nm to precisely match wavelength of the illuminating device to the HbO2 absorption maxima and reduce off-absorption band interferences to the signal. Use of the narrow band interference filters improves image quality and reduces interference associated with the larger bandwidth of the one or more illuminating devices and their mismatch, if any, with HbO2 absorption maxima. Further, the control unit transmits the one or more images captured and the live video image to a computing device connected to the HBM device.

Initially, the HBM device is calibrated by capturing one or more images of the diffuse reflectance for different illuminating sources of light and from a dark background target positioned at the focal plane. Upon capturing the one or more images, the HBM device is used to screen for suspicious lesions with 405 nm illumination for obtaining a live video. On identification of suspicious lesions via the live video, one or more diffuse reflectance and fluorescence images are captured by sequentially illuminating the lesions with light emitted from multiple LED sources.

The computing device processes the one or more images captured to remove effects due to non-uniform illumination, specular reflection, spherical aberration, etc. and analyses these images to detect the grade of cancer and/or inflammation in the tissue. Diffuse reflectance (DR) image ratios (R545/R575, R610/R545 and R610/R575) are computed from the processed images and are Pseudo Colour Mapped (PCM) and displayed in real time to provide an improved and clear visualization of abnormalities in the tissue. The most malignant site in the lesion coincides with the maximum value of the R545/R575 ratio as displayed in the PCM image, and gets represented as the Region Of Interest (ROI). The mean pixel intensity of the ROI is further used in a scatter plot to correlate with histopathological results of biopsy using a diagnosing algorithm that assess level of malignancy and inflammatory status of the tissue. Further, an increase in the R610/575 ratio also serves as an indicator of the grade of the tissue inflammation. Further, the present disclosure includes superimposition of one or more images of tissue fluorescence and diffuse reflectance ratios to reduce false diagnosis and improve accuracy in detecting the grade of cancer and the grade of inflammation. The HBM device disclosed in the present disclosure is non-invasive, as a result of which optical technologies such as those based on autofluorescence and diffuse reflectance have the potential to improve accuracy and availability of cancer screening by interrogating changes in tissue architecture, cell morphology and biochemical composition.

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

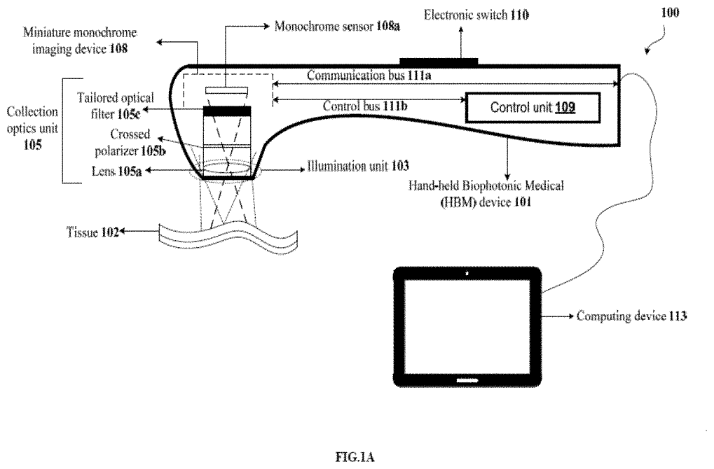

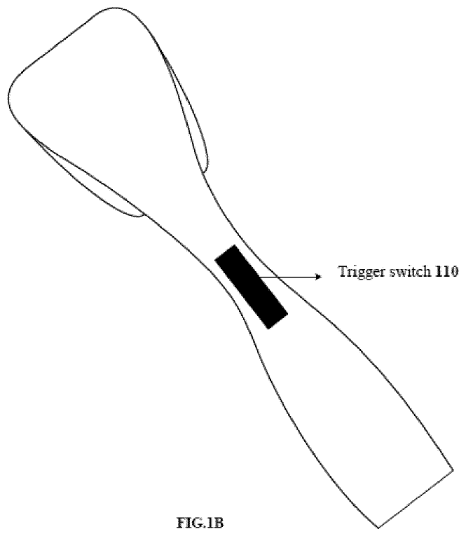

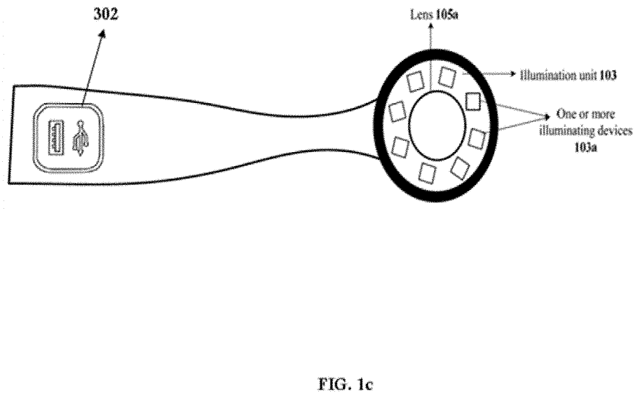

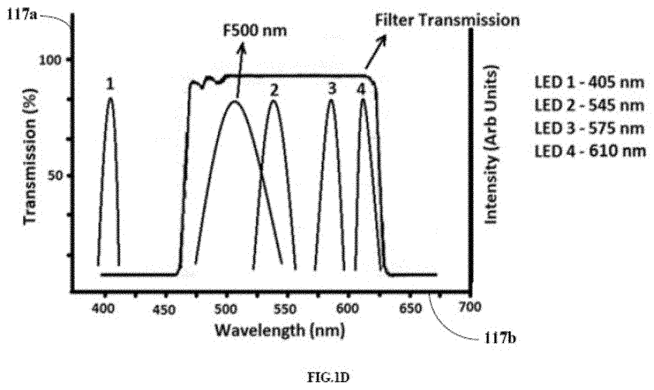

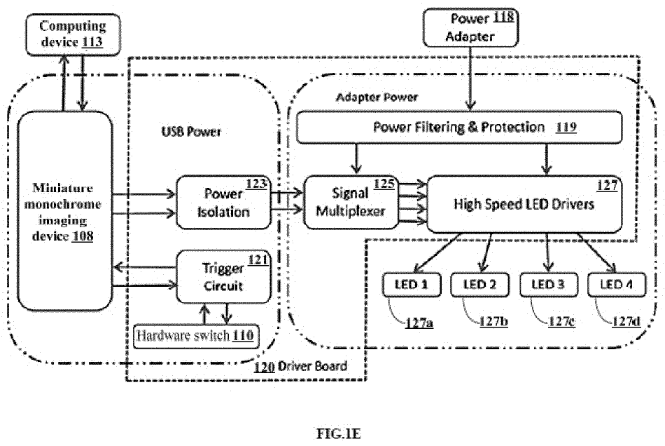

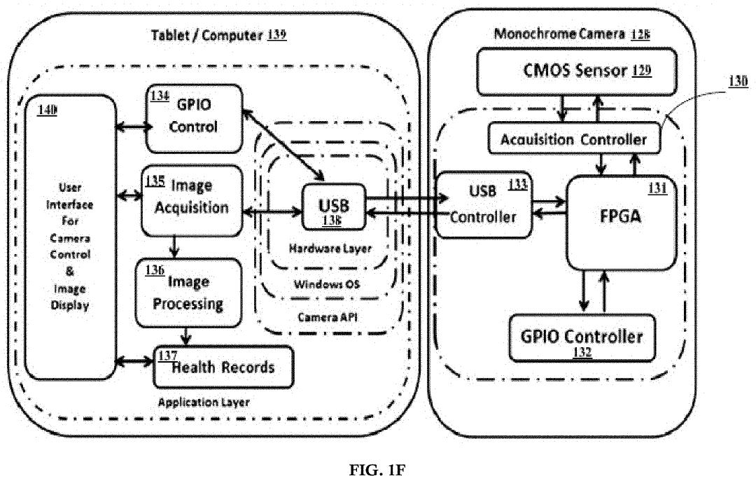

FIG. 1A shows an exemplary system illustrating process for multimodal and multispectral imaging of a tissue in accordance with some embodiments of the present disclosure in accordance with some embodiments of the present disclosure. The system 100 includes a Hand-held Biophotonic Medical (HBM) device 101, a tissue 102 and a computing device 113. The HBM device 101 is connected to the computing device 113 via a wired communication network. In some embodiments, the HBM device 101 may be associated with the computing device 113 via wireless communication networks. As an example, the computing device 113 may include, but not limited to a mobile, a tablet, a laptop and a desktop. In some embodiments, the computing device 113 is configured with a display screen (not shown in the FIG. 1A). In some other embodiments, the computing device 113 may be associated with a display device (not shown in the FIG. 1A), if the computing device 113 is not configured with the display screen.

In some embodiments, as shown in the FIG. 1A, the HBM device 101 comprises an illumination unit 103, a collection optics unit 105 comprising a lens 105a, a crossed polarizer 105b and a tailored optical filter 105c, a miniature monochrome imaging device 108, a monochrome sensor 108a, a control unit 109, an hardware switch 110, a communication bus 111a and a control bus 111b.

In some embodiments, when the HBM device 101 is powered on, an illuminating device 103a of the illumination unit 103 may be emitting at 405 nm suitable for inducing Protoporphyrin IX (PpIX) or FAD fluorescence from tissues or may be emitting at 365 nm suitable for inducing collagen fluorescence. Further, the miniature monochrome imaging device 108 associated with the HBM device 101 streams the live video of the tissue fluorescence to a computing device 113 associated with the HBM device 101, when the tissue 102 is illuminated by the illuminating devices 103a emitting at 405 nm. Upon displaying the live video by the computing device 113, one or more trigger pulses may be generated based on requirement such as when a tissue abnormality is detected in the live video. In some embodiments, the one or more trigger pulses may be generated by manually triggering the hardware switch 110 provided on the handheld biophotonic device or by using the computing device 113 connected to the biophotonic device through wired or wireless connection.

In some embodiments, the hardware switch 110 is located on upper body of the HBM device 101 as shown in the FIG. 1B that shows a top view of the HBM device 101. In some embodiments, the hardware switch 110 may be located at any other place on the HBM device 101. The hardware switch 110 may be at least one of hard buttons and touch screen icons. In some alternative embodiments, the one or more trigger pulses may be generated by the computing device 113. The one or more trigger pulses generated may be transmitted to the control unit 109 via the control bus 111b.

In some embodiments, the control unit 109 receives the one or more trigger pulses generated by the hardware switch 110 or the computing device 113 via the control bus 111b. Upon receiving the one or more trigger pulses, the control unit 109 activates the illumination unit 103. In some embodiments, activating the illumination unit 103 includes sequentially triggering one or more illuminating devices 103a configured within the illumination unit 103. In some embodiments, the one or more illuminating devices 103a may include, but not limited to, one or more Light Emitting Diodes (LEDs). Each of the one or more illuminating devices 103a emit at one or more predefined wavelengths with predefined bandwidths. As an example, if the HBM device 101 is used for examining the oral cavity, the one or more illuminating devices 103a may be the one or more LEDs emitting at, but not limited to, one or more predefined wavelengths of 405 nm, 535 nm, 580 nm and 610 nm and emission bandwidth Full Width Half Maximum (FWHM) of 20-30 nm. In some embodiments, the one or more illuminating devices 103a are arranged in a circular pattern in the illumination unit 103 and a USB port 302 is provided as shown in the FIG. 1C. Further, in some embodiments, the one or more illuminating devices 103a positioned at diametrically opposite locations within the circular arrangement of the illumination unit 103 may be of the same predefined wavelength and the same predefined bandwidth to achieve uniform illumination of the tissue 102. Furthermore, in some embodiments, light from the one or more illuminating devices 103a positioned within the circular arrangement of the illumination unit 103 may be passing through narrowband interference filters of predefined wavelength and bandwidth to match the absorption of targeted absorbers in the tissue 102. As an example, in case of oral cancer detection, the narrowband interference filters of 8±2 nm bandwidth (FWHM) centered at 546 nm, 578 nm and 610 nm (±2 nm) may be used to precisely match the predefined wavelength of the one or more illuminating devices 103a with oxygenated hemoglobin absorption peaks and its off-absorption wavelength. Further, in case of cervical cancer detection, the illuminating device emitting at the predefined wavelength of 405 nm may be replaced with another illuminating device emitting at 365 nm to match absorptions peaks of Collagen. In some embodiments, the narrowband interference filters may be fixed on acrylic glass window at front end of the device 103a. In some embodiments, each of the one or more illuminating devices 103a may be associated with a polarizer (not shown in the figures) such that light emitted from the each of the one or more illuminating devices 103a passes through the polarizer to obtain the light of a particular polarization.

Furthermore, upon receiving the one or more trigger pulses, the control unit 109 activates the miniature monochrome imaging device 108 integrated within the HBM device 101. As an example, the monochrome miniature imaging device 108 may be a miniature monochrome Universal Serial Bus (USB) camera. The miniature monochrome imaging device 108 comprises a monochrome sensor 108a that converts light waves into electrical signals that represent the captured images. As an example, the monochrome sensor 108a may be a Complementary Metal-Oxide-Semiconductor (CMOS) sensor, a Charge-Coupled Device (CCD) sensor and the like. In some embodiments, when the one or more illuminating devices 103a illuminate the tissue 102, the miniature monochrome imaging device 108 may capture one or more images of the tissue fluorescence upon absorption of the light by constituents of the tissue 102 and diffusely reflected light due to multiple elastic scattering of the incident light in the tissue 102 in real-time. As an example, the tissue fluorescence may be captured when the illuminating device 103a having the predefined wavelength of 405 nm or 365 nm with a predefined bandwidth illuminates the tissue 102.

In some embodiments, the following series of actions occur upon activating the illumination unit 103 and the miniature monochrome imaging device 108.

The one or more illuminating devices 103a may be sequentially triggered upon activating the illumination unit 103. The incident light emitted by the one or more illuminating devices 103a passes through the polarizer associated with the one or more illuminating devices 103a. The incident light passing through the polarizer illuminates the tissue 102 with light of a particular polarization. The incident light of particular polarization is absorbed by constituents of the tissue 102. As an example, the constituents of the tissue 102 may be Flavin Adenine Dinucleotide (FAD), Porphyrins, NADH, collagen, protoporphyrin IX, bacteria and their emissions and the like. In some embodiments, the absorption of the incident light of the particular polarization produces the tissue fluorescence. Further, the incident light may be diffusely reflected due to multiple elastic scattering in the tissue 102. Furthermore, the tissue fluorescence and the diffusely reflected light passes through the collection optics unit 105. The lens 105a is positioned within the collection optics unit 105 as shown in the FIG. 1A. The lens 105a collects the tissue fluorescence and the diffusely reflected light from the tissue 102 and directs towards the tailored optical filter 105c via the crossed polarizer 105b. As an example, the tailored optical filter 105c may be a tailored broadband interference filter. In some embodiments, the crossed polarizer 105b is positioned between the lens 105a and the tailored optical filter 105c in a crossed position. The crossed polarizer 105b minimizes/removes specular reflection component in the diffusely reflected light.

In some embodiments, upon receiving the tissue fluorescence and the diffusely reflected light from the lens 105a, the tailored optical filter 105c transmits light of a predefined wavelength range (also referred to as one or more predefined wavelengths) that matches the tissue fluorescence and the diffusely reflected light to the monochrome sensor 108a. The tailored optical filter 105c is constructed such that only the light of the predefined wavelength range passes through the tailored optical filter 105c. As an example, the predefined wavelength range of the tailored optical filter 105c may typically be 475-615 nm (at FWHM) if the absorption is related to the tissue constituents such as FAD, porphyrin and NADH that emit fluorescence in the predefined wavelength range. As an example, if the absorption is related to collagen and other tissue absorbers at 365 nm in cervical tissues, the tailored optical filter 105c may have a transmission in the 420-615 nm range (at FWHM). Exemplary transmission characteristics of the tailored optical filter 105c and emission characteristics of the one or more illuminating devices 103a i.e. LEDs emitting at 405 nm, 545 nm, 575 nm and 610 nm of the illumination unit 103 of the HBM device are shown in the FIG. 1D. In the FIG. 1D, X-axis 117b represents Wavelength in nanometre (nm) and Y-axis 117a represents transmission of the tailored optical filter 105c in percentage. As an example, during screening of the tissue 101, fluorescence emission from the tissue constituents at 500 nm may be allowed to pass through the tailored optical filter 105c, while the emitted light of 405 nm that induces the fluorescence emission from the tissue 101 is completely blocked from reaching the miniature monochrome imaging device 108. Further, the tissue fluorescence and the diffusely reflected light transmitted by the tailored optical filter 105c are received by the monochrome sensor 108a. The miniature monochrome imaging device 108 may capture the one or more images by converting the tissue fluorescence or the diffusely reflected light in the predefined wavelength ranges into electrical signals due to photoelectric effect in the monochrome sensor 108a. In some embodiments, the live video may be streamed at low resolution and higher frame rate and the one or more images may be captured at high resolution. Further, the miniature monochrome imaging device 108 transmits the one or more images to the computing device 113 via the communication bus 111a.

In some embodiments, the computing device 113 may receive the one or more images from the HBM device 101. In some embodiments, the computing device 113 may be installed with an image processing application combined with a diagnosing algorithm. In some embodiments, the diagnosing algorithm is a machine learning algorithm. The computing device 113 may analyze the one or more images using the image processing application to detect changes in intensity of oxygenated hemoglobin and other absorbers in the tissue 102 at the predefined wavelength range. Further, the computing device 113 generates one or more pseudo colored images of the one or more images received by the computing device 113. The one or more pseudo colored images are obtained by false coloring the one or more images. False coloring the one or more images provides a clear visualization of abnormalities in the tissue 102. Furthermore, the computing device 113 determines image intensity ratio values of the one or more images captured by the miniature monochrome imaging device 108. As an example, consider the one or more images of the diffusely reflected light captured at the predefined wavelength range 545 nm, 575 nm and 610 nm by the miniature monochrome imaging device 108. Therefore, the image intensity ratio values may be computed as R545/R575, R610/R575 and R610/R545. Upon determining the image intensity ratio values, the computing device 113 may identify Regions of Interest (ROI) comprising a maximum change in the image intensity ratio values when compared to a predefined standard ratio value obtained from normal/healthy tissues of similar anatomical sites. In some embodiments, the predefined standard ratio value is related to the ROI of a similar (corresponding) site in a normal healthy tissue. As an example, tissues in the oral cavity may show dips at 545 nm and 575 nm due to absorption by oxygenated haemoglobin. The image intensity ratio value R545 nm/R575 nm is lowest for the normal healthy tissue in the oral cavity. Therefore, a high image intensity ratio value of R545 nm/R575 nm is considered as the maximum change when compared to the image intensity ratio value in the normal healthy tissue. The computing device 113 determines at least one of grade of cancer or a grade of inflammation in the tissue 102 automatically based on the intensity of the oxygenated haemoglobin absorption and by correlating the image intensity ratio values obtained from the one or more images using a diagnosing algorithm. The diagnosing algorithm correlates the image intensity ratio values with pathological reports of tissue biopsy from the same site. As an example, decrease in the image intensity ratio value R545 nm/R575 nm at a particular ROI or a increase in the image intensity ratio value R610/R575 at the same ROI may indicate an inflammatory condition of the tissue 102. Therefore, the diagnosing algorithm may determine grade of inflammation based on the amount of increase or decrease in the image intensity ratio values. Further, the computing device 113 may superimpose at least one of the one or more images or their image intensity ratio values to reduce false diagnosis of the tissue 102. As an example, the image intensity ratio value R545/R575 may be superimposed on the tissue fluorescence image to increase accuracy in detecting the grade of cancer and grade of inflammation. Further, the computing device 113 may store information related to a patient being diagnosed using the HBM device 101. As an example, the information related to the patient may include, but not limited to, name of the patient, age of the patient, sex of the patient, medical condition of the patient and the determined grade of inflammation or grade of cancer of the patient.

FIG. 1E shows internal architecture of the system for multimodal and multispectral imaging of a tissue in accordance with some embodiments of the present disclosure. Each block of represented in the FIG. 1E should be considered as a unit block.

The internal architecture comprises the unit block “power filtering and a protection 119” that activates a Hand-Held Biophotonic Medical (HBM) Device 101 by supplying power that is filtered according to requirement of the HBM device 101. In some embodiments, the power is received from a power adaptor 118 associated with the HBM device 101. In some embodiments, the power adaptor 118 may be replaced with a portable battery bank for operating the HBM device 101 even in remote areas without electricity. Further, the unit block “power filtering and protection 119” includes electrostatic discharge and under/over current protection features that protects the HBM device 101 from external power fluctuations. Furthermore, the unit block “power isolation 123” isolates the power using a digital-optical isolation Integrated Circuit (IC), that in turn protects both a miniature monochrome imaging device 108 and a driver board 120 from internal power variations integrated in the HBM device 101. Further, the unit block “signal multiplexer 125” is configured to split two bits signals received from the unit block “power isolation 123” to four analog signals. Each of these four analog signals is used to switch the unit block “high speed Light Emitting Diode (LED) drivers 127”. The unit block “high speed LED drivers 127” comprises a Metal-Oxide-Semiconductor Field-Effect Transistor (MOSFET) based switching circuit that provides high speed switching. Further, the unit block “high speed LED drivers 127” also comprises a variable resistor for fine tuning output power of the LEDs such as LED1 127a, LED2 127b, LED3 127c and LED4 127d and a current limiting resistor for protection of the LEDs. Further, the internal architecture comprises the unit block “trigger circuit 121” that is configured to generate one or more trigger pulses as input to the miniature monochrome imaging device 108, that in turn communicates with a computing device 113.

FIG. 1F shows an exemplary application layer of miniature monochrome imaging device and a computing device in accordance with some embodiments of the present disclosure.

In the FIG. 1F, a miniature monochrome camera 128 comprising a monochrome Complementary Metal-Oxide-Semiconductor (CMOS) sensor 129 featuring a high frame rate (30 fps) with high-speed data transfer via Universal Serial Bus [USB] 2.0 is represented. The General-Purpose Input/Output [GPIO] controller 132 of the miniature monochrome camera 128 controls the Light Emitting Diode (LED) switching via a hardware trigger. These functionalities are performed by the Field-Programmable Gate Array [FPGA] 131 inside the miniature monochrome camera 128. Further, the miniature monochrome camera 128 is controlled by an application running on a Tablet/computer 139. The Application Programming Interface (API) communicates with the miniature monochrome camera 128 via a USB Driver 133 of the computer 139. The FPGA 131 has a built in USB Controller 133 that is configured to maintain communication of the miniature monochrome camera 128 with the computer 139. The application has the capability for streaming a live video and capture and display captured images on a user interface/display interface 140 of the computer 139. It controls the GPIOs, processes the one or more images with suitable algorithms and manages patient health records.

The application layer is responsible for the operation of the Hand-held Biophotonic Medical Device (HBM) device 101. Following are steps to be executed by the application.
1. Collect and store patient information.
2. Imaging process
   a. Send command to GPIO controller 132 in the FPGA 131 and turn on respective port.
   b. Send command to acquisition controller 130 in the GPIO to grab frame in low resolution.
   c. View the live frames in the window of the application.
   d. On hardware trigger the GPIO controller 132 will send signal to FPGA 131
   e. FPGA 131 will send signal to application via USB 133.
   f. Application will start initiating the multispectral imaging process with respect to signal.
   g. The application will send the command to GPIO controller 132 in the FPGA 131
      and turns on the respective port.
   h. Further, the application will send command to the acquisition controller 130 in the GPIO to grab frame in high resolution.
   i. This process is repeated in accordance with imaging sequence.

Further, the image acquisition 135 and image processing 136 parts of the application correct the one or more images for lens aberration. Further, the one or more images are pseudo colour mapped for determining pixel intensity values from Region Of Interest (ROI), that are in turn compared by a diagnosing algorithm present in the computer 139 with pathology. Finally, the electronic health records 137 are stored along with images and the patient information that are further secured by transmitting them to cloud storage.

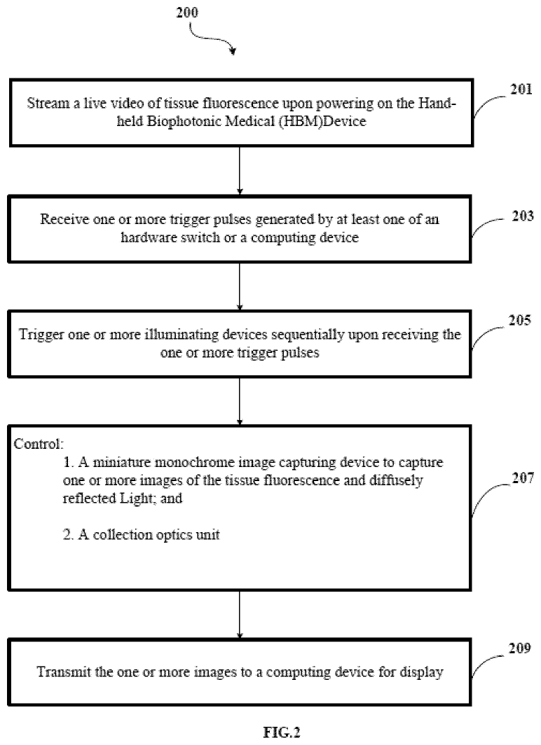

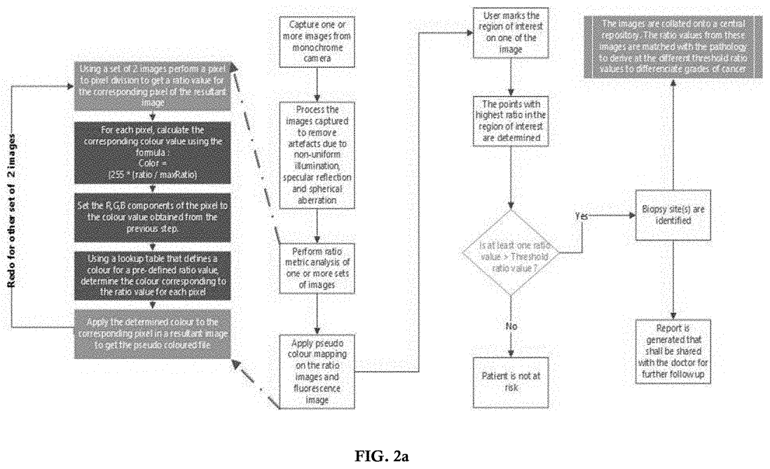

FIG. 2 shows a flowchart illustrating a method for multimodal and multispectral imaging of a tissue in accordance with some embodiments of the present disclosure.

As illustrated in FIG. 2, the method 200 includes one or more blocks illustrating a method for providing gesture-based interaction with a virtual product. The method 200 may be described in the general context of computer executable instructions. Generally, computer executable instructions can include routines, programs, objects, components, data structures, procedures, modules, and functions, which perform functions or implement abstract data types.

The order in which the method 200 is described is not intended to be construed as a limitation, and any number of the described method blocks can be combined in any order to implement the method 200. Additionally, individual blocks may be deleted from the methods without departing from the spirit and scope of the subject matter described herein. Furthermore, the method 200 can be implemented in any suitable hardware, software, firmware, or combination thereof.

At block 201, the method 200 may include streaming, by a Hand-held Biophotonic Medical (HBM) device 101, a live video of tissue fluorescence. In some embodiments, the HBM device 101 is powered on manually. Upon powering on the HBM device 101, by default, an illuminating device 103a emitting at a fluorescence inducing wavelength such as 405 nm is activated. Further, a miniature monochrome imaging device 108 associated with the HBM device 101 streams the live video of the tissue fluorescence to a computing device 113 associated with the HBM device 101, when a tissue 102 is illuminated by the illuminating device 103a emitting at 405 nm or at 365 nm. At block 203, the method 200 may include receiving, by the HBM device 101 one or more trigger pulses from a hardware switch 110 of the HBM device 101. In some embodiments, the one or more trigger pulses may be generated when the hardware switch 110 is triggered manually.

In some alternative embodiments, the one or more trigger pulses may be generated by the computing device 113. The one or more trigger pulses may be generated based on requirement such as when a tissue abnormality is detected upon viewing the live video.

At block 205, the method 200 may include triggering, by the HBM device 101, one or more illuminating devices 103a sequentially to illuminate the tissue 102 upon receiving the one or more trigger pulses. The incident light pulses emitted by the one or more illuminating devices 103a passes through a polarizer and narrowband interference filters associated with the one or more illuminating devices 103a. The incident light passing through the polarizer and interference filter illuminates the tissue 102 with light of a particular polarization. The incident light of particular polarization is absorbed by constituents of the tissue 102 to generate tissue fluorescence and also undergo multiple elastic scattering and absorption by HbO2 in the tissue 102 to generate diffusely reflected light.

At block 207, the method 200 may include controlling, by the HBM device 101, a miniature monochrome image capturing device 108 associated with the HBM device 101 to capture one or more images of the tissue fluorescence and the diffusely reflected light in real-time using the miniature monochrome imaging device 108 and a collection optics unit 105 associated with the HBM device 101 upon receiving the one or more trigger pulses. In some embodiments, the collection optics unit 105 includes, but not limited to, a lens 105a, a crossed polarizer 105b and a tailored optical filter 105c. In some embodiments, one or more lenses may be present in the HBM device 101. The lens 105a collects the tissue fluorescence and the diffusely reflected light from the tissue 102 and directs the collected light through the tailored optical filter 105c via the crossed polarizer 105b. In some embodiments, the crossed polarizer 105b is positioned between the lens 105a and the tailored optical filter 105c in an orthogonal orientation with respect to the polarizer positioned in front of the one or more illuminating devices 103a to minimize/remove specular reflection component in the diffusely reflected light. The tailored optical filter 105c transmits light of a predefined wavelength range (also referred to as one or more predefined wavelengths) that matches the tissue fluorescence and the elastically scattered light from LEDs emitting at 545, 575 and 610 nm to the monochrome sensor 108a. Using the light in the predefined wavelength range, the miniature monochrome imaging device 108 captures the one or more images in a high resolution.

At block 209, the method 200 includes transmitting, by the HBM device 101, the one or more images to a computing device 113 connected with the HBM device 101 for further processing and display. In some embodiments, the processing involves correction of the light incident on the sensor for non-uniform illumination and extraction of diffusely reflected light using light reflected by one or more light sources from a reflectance standard. In some embodiments, the computing device 113 displays the live video and the one or more images in real-time.

The method 200 further comprises capturing a background image of a lesion under ambient light without illuminating the tissue and subtracting said background image from the images of illuminated tissue.

FIG. 2a elucidates a flow diagram for the image acquisition 135 and image processing 136 parts of the application given in the present invention. The process of image acquisition 135 and image processing 136 comprises of:
a) capturing one or more images from the miniature monochrome camera 128;
b) processing the images captured in step a) to remove effects due to the presence of background light and artifacts due to non-uniform illumination, specular reflection and spherical aberration;
c) performing pixel to pixel subtraction for a set of at least two images for removing background light and ratio metric analysis of one or more sets of images processed in step b);
d) performing a pixel to pixel division of a set of at least two images from step c) for getting a image intensity ratio value for the corresponding pixel of the resultant image;
e) calculating a corresponding colour value for each pixel after division in step d);
f) setting the R, G, B components of the pixel to the colour value obtained in step e);
g) determining a colour corresponding to the image intensity ratio value for each pixel obtained in step d) using a look up table that defines a colour for a predefined ratio value;
h) applying the colour determined in step g) to the corresponding pixel in a resultant image for getting a pseudo coloured file;
i) applying pseudo colour mapping on the image intensity ratio images and fluorescence images;
j) marking at least one region of interest on one of the pseudo colour mapped image in step i);
k) determining points with highest image intensity ratio in the region of interest marked in step j);
l) comparing the image intensity ratio obtained in step k) with a predefined threshold ratio value;
m) identifying biopsy site(s) if the at least one of the image intensity ratio obtained in step k) is greater than threshold ratio value after comparison at step 1);
n) carrying out pathology from the identified biopsy site in step m);
o) collating images captured in step a) onto a central repository;
p) matching the ratio values from the collated images in step o) with the pathology in step n) for deriving the different threshold values for differentiating grades of cancer; and
q) generating and sharing a report with a doctor for treatment.

In step e) a corresponding colour value for each pixel is calculated using equation (1):

Colour=(255*(Ratio/maxRatio)) (1)

Ratio is ratio value obtained for the corresponding pixel of the resultant image after performing a pixel to pixel division in step d); and

maxRatio is the maximum ratio obtained out of the ratio value obtained for the corresponding pixel of the resultant image.

The central repository is either a central server or internet cloud to store all the captured images, diagnostic algorithms and reports which are shared with doctors for further follow up and treatment.

In some embodiments, the computing device 113 may analyze the one or more images using the image processing application to detect changes in absorption intensity of oxygenated hemoglobin in the tissue 102 at the predefined wavelength range. The oxygenated hemoglobin has absorption maxima typically around 543 nm and 577 nm. The haeme cycle is disturbed in malignant tissues due to the reduced activity of ferro chelatase enzyme leading to a selective accumulation of protoporphyrin IX (PpIX) and lower production of hemoglobin in the tissue 102. The accumulation of PpIX and the low production of hemoglobin introduces absorption anomalies in the oxygenated hemoglobin spectra that help in detecting presence of malignancy in the tissue 102 from the ratio of the one or more images captured at 545 nm, 575 nm and 610 nm.

Further, the computing device 113 generates one or more Pseudo Color Mapped (PCM) images by false coloring the one or more images or their ratio images. Further, the computing device 113 determines at least one of a grade of cancer or a grade of inflammation in the tissue 102 automatically based on the intensity of the oxygenated haemoglobin absorption and by correlating the image intensity ratio values at the ROI obtained from the one or more images using a diagnosing algorithm. As an example, the grade of cancer may be assigned based on whether the tissue 102 is determined to be poorly differentiated, moderately differentiated, well differentiated, dysplastic, hyperplastic and the like. As an example, the grade of inflammation may be minimal, mild, moderate, severe and the like. In some embodiments, the computing device 113 may perform superimposing at least one of the one or more images or the determined image intensity ratio values to reduce false diagnosis of the tissue 102. Further, the computing device 113 may store information related to a patient being diagnosed using the HBM device 101. In some embodiments, the patient may be, but not limited to, human beings. As an example, the information related to the patient may include, but not limited to, name of the patient, age of the patient, sex of the patient, medical condition of the patient and the determined grade of inflammation or grade of cancer of the patient.

The present invention provides a method for multi-spectral screening and detection of oral potentially malignant disorders non-invasively by fluorescence and diffuse reflectance imaging with the hand held biophotonic device. The most malignant site in a lesion for biopsy is identified by processing of the captured images. The pathological results of biopsy taken from the most malignant site is correlated with the diffuse reflectance image ratios (R610/R545 and/or R545/R575) to develop an algorithm for tissue discrimination between different grades of cancer and to determine the sensitivity and specificity of tissue classification. The algorithm thus developed incorporating a large number of data sets would be useful to have an idea on the grade of cancer in real time at the point-of-care.

Biochemical, morphological and structural changes occur during tissue transformation towards malignancy, which are studied from changes in tissue fluorescence, absorption and scattering. When light enters a tissue, various optical processes such as scattering (elastic and inelastic scattering), absorption and emission take place. The handheld biophotonic device detects changes in fluorescence intensity across the oral mucosa due to changes in biochemical constituents of tissue such as NADH, FAD, collagen and Protoporphyrin IX (PpIX) on excitation with 405 nm LED light. It also monitors changes in oxygenated hemoglobin (HbO2) concentration from the absorption intensity of its characteristics peaks located at 545 and 575 nm and from a wavelength (605 nm) that does not contribute to HbO2 absorption.

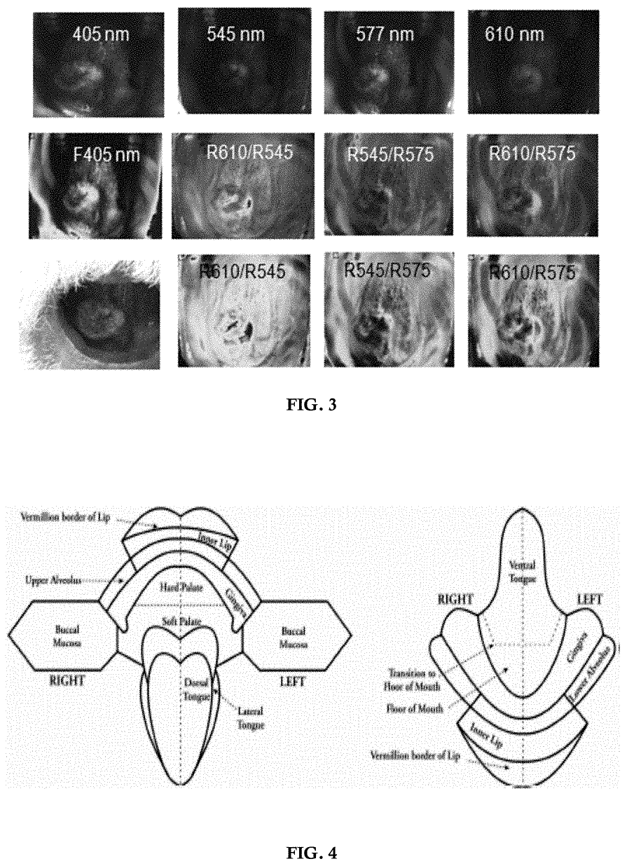

In cancer cells, the heme synthesis is disturbed due to the reduced activity of the ferro chelatase enzyme that results in PpIX increase and lowering of hemoglobin production and correspondingly lower absorption of HbO2 at 542 and 577 nm. A reduction in HbO2 increases the DR ratio (R545/R575 and R610/R545) in malignant tissues. Conversely, during inflammatory conditions there is an increase in heme production, which leads to an enhancement in the oxygenated hemoglobin and a concomitant decrease in the DR image ratio of R545/R575 and also an increase in the R610/R575 ratio. The monochrome camera of the handheld device captures both the tissue fluorescence in the 470-610 nm, and DR images at 545, 575 and 610 nm. The proprietary software program run on tablet connected to the device computes the ratio R545/R575, R610/R545 and R610/R575 and presents the pseudo colour map (PCM) images of these ratios and the fluorescence image for identification of tissue abnormalities in real time. The present multimodal imaging device utilizes a combination of tissue auto fluorescence (AF) and DR imaging at the oxygenated hemoglobin absorption peaks to screen and detect OPMD lesions, and to discriminate malignant sites from normal and inflammatory tissues. In FIG. 3, the top row shows a set of monochrome images captured by the device on illumination at 405 nm, 545 nm, 575 nm and 610 nm. The middle row shown the pseudocolor map of fluorescence image is F405, whereas the R610/R545, R545/R575 and R610/R575 on the same row are ratio images derived from the captured monochrome images after background subtraction and image division. The last row shows a photograph of the cancer lesion (well differentiated squamous cell carcinoma) on the floor of the mouth of the patient and the pseudo color maps of the monochrome image ratios shown in the middle row, highlighting areas affected by cancer or tissue inflammation. The most malignant site in the lesion can be easily identified for tissue biopsy from the PCM of the ratio images. The clinical validation studies carried out points to the potential of the device to identify the optimal site in a lesion for biopsy, thereby reducing the large number of false negatives, multiple biopsies, late stage diagnosis and treatment costs.

### EXAMPLE 1

### Utilization for Hand-Held Biophotonics Device for Detection of Oral Potentially Malignant Disorders

Oral squamous cell carcinoma (OSCC) remains a significant health burden across the globe despite commendable progress in the screening and detection of oral cancers. In clinical practice, opportunities exist to identify patients with oral potentially malignant disorders (OPMDs), which precede the development of cancer, such as leukoplakia, erythroplakia, and oral submucous fibrosis (OSF), to a limited extent. Before practically using the handheld device, it needs to be calibrated and validated. Hence, to utilize the handheld device as a screening tool for early detection oral cancer, a study covering a large population following a standard calibration and validation methodology, with facilities for information storage, retrieval and utilization is essential.

Study Population:

When the disease status is known, the formula for sample size for diagnostic tests, with (1−α) % confidence level and with maximum marginal error (precision) of estimate (d), for constructing confidence interval with true value of sensitivity (or specificity) using normal approximation is given in equation (1)

N=Z²P(1−P)/d² (1)

Where P is the pre-determined value of sensitivity (or specificity) that is ascertained from previously published data or clinician experience; and for α=0.05, Z is inserted as 1.96.

Therefore, for achieving a sensitivity and specificity of 95%, with marginal error of 0.1 and disease prevalence d=0.5 in target population, the number of subjects required is 36. Further, we need to discriminate tissue into 4 groups such as normal, hyperplasia, dysplasia (pre-cancer lesions consisting of mild, moderate, severe dysplasia and carcinoma in situ (CiS)), and SCC (Well- moderately- Poorly differentiated SCC). This would require 108 patients (36×3) and, a control group of 36 healthy subjects (with no previous history on usage of commercial preparations such as tobacco, cigarette, pan, gutka or alcohol) to develop statistically significant algorithm. Thus, a total of 150 subjects would be required to complete the algorithm development. Further, 50 patients would be required to test the algorithm developed and determine the diagnostic accuracy.

Methodology:

Patients are initially examined by a clinician with torch light to detect any abnormal lesions in the oral cavity. On powering the handheld biophotonic device, the violet light (405 nm) comes on, which is useful as a screening tool for tissue abnormalities in the live-view mode. Before initiation of measurements, the device is calibrated using a tissue phantom. The software has provision to fine tune the gain and exposure settings of the camera to suit the low (partially dark) ambient light conditions, which is required for capturing of tissue fluorescence and the diffusely reflected light from oral cavity tissues. Afterwards, the oral cavity of the patient is examined in live view mode on the tablet with 405 nm illumination of the handheld biophotonic device. The abnormal areas of the oral cavity are noted from changes in tissue fluorescence.

The camera is focused to obtain a clear view of the suspicious site and multispectral images of tissue fluorescence and diffusely reflectance on illumination with different LEDs emitting at 405, 545, 575 and 610 nm are captured using the trigger switch on the device or the capture button on the software. The recorded images are then processed and the fluorescence and DR image ratios (R545/R575, R610/R575 and R610/R545) are computed and displayed in the software panel along with the raw monochrome images. Regions of interest (ROI) are then marked on images and the software locates and marks sites with the highest values of the ratios in the RoI, which can be saved along with the captured images. If the images captured are not good enough, the software has provision to discard these images and recapture another set of images. On completion of the screening procedure the recorded ratio values are examined to understand the severity of the device and decide on whether a follow up is required. The processed images after pseudo color mapping (PCM) will help the clinician to identify the most malignant site in the lesion for biopsy. In the case of healthy subjects, no biopsy is taken; but the clinician would visually examine the patient and confirm that the oral cavity is apparently healthy. In cases that require a biopsy, an incisional or a punch biopsy is taken from the most malignant site identified by the device. The histopathological result of biopsy is then correlated with the image ratio values (R610/R545 and R545/R575) and an algorithm is developed to discriminate different grades of cancer. The ratio values corresponding to healthy subjects screened will also form part of these algorithms, which can be used to determine the diagnostic accuracy for discrimination between different grades of cancer.

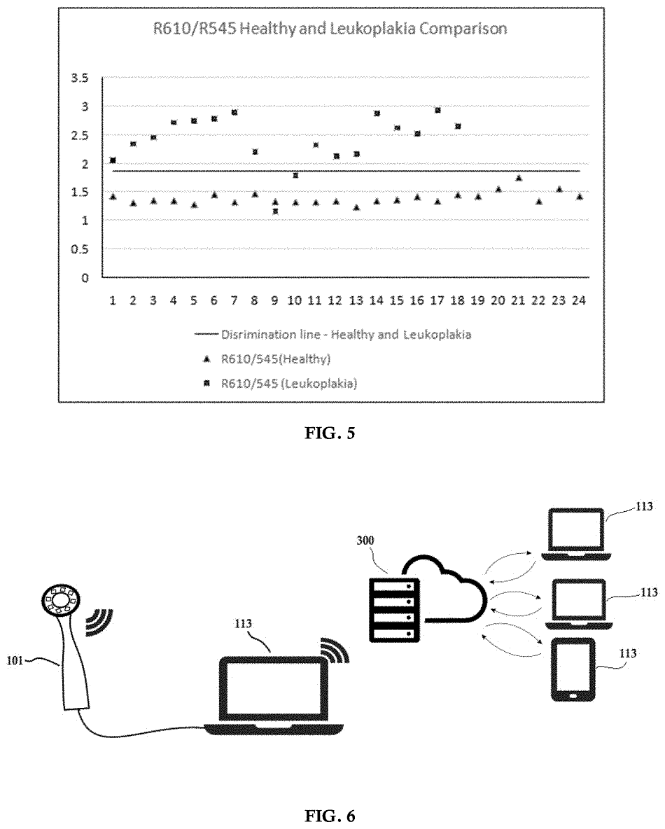

FIG. 5 shows a scatter plot diagram discriminating leukoplakia from healthy tissues of the oral mucosa using the R610/R545 image ratio. The discriminant lines are drawn at the mean of the ratio value between adjoining categories, which are then used to determine the diagnostic accuracy for discrimination between the two respective grades of cancer.

Patient Data/Information Storage:

The handheld biophotonic device has an information input interface such as GUI and a keypad of a computing device such as a tablet or personal computer to enter all patient details and visual impression of the disease. It also has an interface to mark the site identified by the clinician based on visual impression. The captured image data is stored in the computing device with all relevant details and the RoI values gets saved in the corresponding ratio image. The image ratios are also logged in automatically. The captured images can be accessed and reviewed on the display unit, which helps the doctor to ascertain the image quality and do recapture if required. FIG. 6 elucidates the topological structure of handheld device 101 with the computing unit 113. All captured image data is initially stored in the computing device 113 and later stored in the server 300 for easy access by clinicians and used in algorithm development.

Data Utilization:

The image data collected from population study is incorporated in a data bank, collated and analyzed. The data would be correlated with pathology results of biopsy and utilized for developing an intelligent and robust algorithm to predict/assess the grade of cancer non-invasively in real time during the screening process with the bimodal hand-held device. It is also possible to determine the sensitivity and specificity of the device for discrimination between different grades by correlating the data with pathological results.

### EXAMPLE 2

### Oral Potentially Malignant Disorder Imaging with Handheld Biophotonic Device

Patients with oral potentially malignant disorder (OPMD) lesions such as leukoplakia, erythroplakia, oral sub mucous fibrosis (OSMF), dysplasia and moderate to well differentiate squamous cell cancer are observed. In this study, patients with previous history of cancer treatment, with systemic conditions that contraindicates biopsy and patients with recent oral medication for at least four weeks are excluded.

The database with a sample size of 200 patients and 40 healthy subjects is created. Further, 50 patients are enrolled to test the developed algorithm and to determine the diagnostic accuracy of the screening device. This is a multicentric study covering 4 participating centers. The protocol followed for imaging of OPMD using handheld device comprises of:
a. washing mouth with distilled water;
b. examining visually the mouth with white light for any abnormal lesions;
c. identifying a site for biopsy based on visual impression if an abnormal lesion is found;
d. cleaning the suspicious area or affected lesions with distilled water to remove crusts, if any;
e. drying the cleaned area using forced air, tissue or cotton gauze;
f. capturing a photograph of the lesion;
g. covering the handheld device probe tip and handle with a transparent film or sheath to avoid probe contamination and to maintain hygiene;
h. calibrating the probe using a tissue phantom following the standard procedures in a dark room environment;
i. inserting the covered probe in the oral cavity;
j. screening the lesion with the violet light (405 nm) of the probe;
k. adjusting the device position to get focused images of the lesion;
l. streaming the images on a computing device such as a PC/Tablet attached to the handheld device through wired or wireless means;
m. triggering the image capture by pushing the trigger on the probe or through the computing device to illuminate the lesion with four different LEDs sequentially;
n. sequentially capture the autofluorescence (450-600 nm) and diffuse reflectance images at 545, 575 and 610 nm;
o. mark regions of interest (ROI) on Pseudo colour mapped (PCM) fluorescence image;
p. confirming the tissue inflammation with R610/R575 image ratio;
q. moving the handheld device to view the contra-lateral site and capturing a set of images for comparative evaluation of the tissue characteristics;
r. re-examining the PCM image ratio if abnormal lesions are seen in the DR ratio images (R545/R575 or R610/R545) to locate and identify the most malignant site for biopsy based on the value of the DR ratio, which may also be corroborated by the PCM of the fluorescence image. In cases where site identified by the PCM of DR image ratio and fluorescence images differ, an additional site may be chosen for biopsy;
s. taking biopsy from the visually identified site and the site(s) identified by the device, if it is different from the site identified visually;
t. sending the biopsy samples to a pathologist and obtaining histopathological results and correlating with image ratio (ROI) values corresponding to the biopsy sites and plotting ROI, the scatter plot algorithms representing R610/R545 and R545/R575 ratios with pathology results, which can be used to determine the grade of cancer through blind studies and to evaluate the diagnostic accuracy of measurement

In cases where early lesions are detected, the cost of treatment and complications would be minimal. Furthermore, with the help of the developed algorithm, it would be possible to use the device as a screening tool to detect OPMD of oral cavity in real time and to locate the optimal site for biopsy.

Advantages of the Embodiment of the Present Disclosure are Illustrated Herein.

In an embodiment, the present disclosure provides a Hand-held Biophotonic Medical (HBM) device, a method and a system for multimodal and multispectral imaging of a tissue. The multiple modes included in this disclosure are fluorescence, absorption, scattering and diffuse reflectance.

The HBM device disclosed in the present disclosure is non-invasive, as a result of which optical technologies such as those based on autofluorescence and diffuse reflectance imaging have the potential to improve accuracy and availability of cancer screening by interrogating changes in tissue architecture, cell morphology and biochemical composition.

The present disclosure discloses using Light Emitting Diodes (LEDs) of one or more predefined wavelengths for illuminating the tissue. The use of LEDs instead of other light sources such as white light source, tungsten halogen lamp, mercury-xenon lamp, arc lamp and the like eliminates the need for expensive filters such as liquid crystal tunable filters, acousto-optic tunable filters, filter wheels and the like for wavelength selection.

The present disclosure discloses a low-cost tailored optical filter that transmits light of one or more predefined wavelengths matching with the tissue fluorescence and the diffusely reflected light in the range of oxygenated hemoglobin absorption.

The present disclosure discloses a feature wherein the LEDs emitting light of desired wavelength for fluorescence imaging and diffuse reflectance imaging are automatically triggered to illuminate the tissue. Therefore, as disclosed in few prior arts, manually operating a shutter to illuminate the tissue with the light of desired wavelength while blocking the light of undesired wavelength is avoided, and associated complications eliminated.

The present disclosure discloses a miniature monochrome camera integrated within the HBM device for live viewing of tissue fluorescence and capturing of fluorescence and diffuse reflectance images of tissues.

Generally, premalignancies are characterized by increased nuclear/cytoplasmic ratio, which is assessed by histopathology. An oral lesion that is premalignant at some part may not be malignant at another location. Therefore, biopsy from one location of the lesion cannot be a representative of the entire lesion. Also, the resemblances of tissue inflammation and irritation with premalignant oral mucosal alterations and field cancerous changes are often challenging to understand. Therefore, the present disclosure discloses a machine-learning diagnosing algorithm that helps in easily detecting various grades of cancer such as a most malignant site, a pre-malignant site and the like and tissue inflammation using the diagnosing algorithm, said tissue being present in cavity such as oral cavity, oesophagus, cervix, larynx, pharynx, GI tract, colon and alike

The HBM device is constructed in such a way that it is light weighted, easily hand held, portable, can be easily inserted into parts of a body such as the oral cavity, cervix and the like. Further, the HBM device can be adapted for use on endoscopes to examine internal organs of the body.

The present disclosure provides a feature wherein the one or more images are pseudo colour mapped before analysing, thus providing a better and clear visualization of tissue abnormalities in real time. Further, the present disclosure includes superimposing one or more images to reduce false diagnosis and improve accuracy in detecting grade of cancer and inflammation.

The HBM device disclosed in the present disclosure is used for screening for oral and cervical cancers that reduces unwanted biopsies and helps in identifying the appropriate biopsy site in real time. Enabling the live video image and real-time image capture and processing helps in performing the kind of screening that reduces many false negatives that are common with the present-day screening techniques. Further, the HBM device helps in minimizing the delay in diagnosis and planning of treatment strategies, thereby saving lives of people suffering from squamous cell carcinoma.

A description of an embodiment with several components in communication with each other does not imply that all such components are required. On the contrary a variety of optional components are described to illustrate the wide variety of possible embodiments of the invention.

When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The specification has described a Hand-held Biophotonic Medical (HBM) device, a method and a system for multimodal and multispectral imaging of a tissue. The illustrated steps are set out to explain the exemplary embodiments shown, and it should be anticipated that on-going technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

I claim:

1. A Hand-held Biophotonic Medical (HBM) device (101) for a multimodal and multispectral imaging of a tissue (102), the HBM device (101) comprising:
a) an illumination unit (103) comprising of a combination of one or more illuminating devices (103a) emitting at one or more wavelengths with narrow bandwidths matching absorption of fluorophores and/or oxygenated haemoglobin in the tissue (102);
b) a hardware switch (110) configured to generate one or more trigger pulses when triggered;
c) a collection optics unit (105) comprising a lens (105a), a tailored optical filter (105c) and a crossed polarizer (105b) that minimizes specular reflection present in the diffusely reflected or emitted light from the tissue;
d) a miniature monochrome imaging device (108) comprising at least one monochrome sensor (108a) for capturing images of the tissue (102); and
e) a control unit (109) connected to said hardware switch (110) and said miniature monochrome imaging device (108) via a communication bus (111a) and a control bus (111b);
wherein,
the hardware switch (110) and the control unit (109), together control power of the HBM device (101);
the illumination unit (103) is configured such that one or more illuminating devices (103a) operate separately to emit narrow band light at one or more wavelengths matching absorption of fluorophores and/or oxygenated haemoglobin in the tissue (102);
the hardware switch (110) and the control unit (109), together control wavelength and bandwidth of light emitted by the illumination unit (103);
the one or more illuminating devices (103a) are triggered sequentially to illuminate the tissue (102) resulting in either a tissue fluorescence upon absorption of an incident wavelength by the tissue (102), and/or diffuse reflectance due to multiple elastic scattering of an incident light by the tissue (102);
the fluorescence and/or the diffusely reflected light are transmitted to said miniature monochrome imaging device (108);
the miniature monochrome imaging device (108) captures the tissue fluorescence and/or diffusely reflected light as one or more images and converts said images into electrical signals;
the miniature monochrome imaging collection optics unit (105) is configured to detect the tissue (102);
the device (108) transmits one or more images to a computing device (113) for image processing and display; and
the control unit (109) is configured to control the miniature monochrome imaging device (108) upon receiving one or more trigger signals.

2. The HBM device (101) as claimed in claim 1, wherein, the multimodal imaging comprises detection of one or more modes of light tissue interaction including but not limited to fluorescence, absorption, transmittance, reflectance, diffuse reflectance, elastic scattering, inelastic scattering, photoacoustic and thermal imaging.

3. The HBM device (101) as claimed in claim 1, wherein the illumination unit (103) further comprises a polarizer configured to illuminate the tissue (102) with light of a particular polarization.

4. The HBM device (101) as claimed in claim 1, wherein the crossed polarizer (105b) is configured to reduce specular reflection from the tissue (102).

5. The HBM device (101) as claimed in claim 1, wherein the monochrome sensor (108a) is a Complementary Metal-Oxide-Semiconductor (CMOS) sensor or a Charge-Coupled Device (CCD) sensor.

6. The HBM device (101) as claimed in claim 1, wherein the miniature monochrome imaging device (108) captures the one or more images by converting the tissue fluorescence or the diffusely reflected light into electrical signals due to photoelectric effect in the monochrome sensor (108a).

7. A system for multimodal and multispectral imaging of a tissue (102), the system comprising:
a) a Hand-held Biophotonic Medical (HBM) device (101); and
b) a computing device (113);
wherein,
said HBM device (101) is configured to illuminate the tissue (102) with a light of wavelength and bandwidth matching absorption of biochemical constituents that gets altered during malignant transformations in the tissue resulting in tissue fluorescence and/or diffuse reflectance due to elastic scattering of light; capture one or more images of tissue fluorescence and/or diffusely reflected light transmitted through a tailored filter (105c); and transmit the one or more images to the computing device (113) for display;
the computing device (113) is configured to:
receive the one or more images transmitted by said HBM device (101);
detect changes in intensity of oxygenated haemoglobin absorption in the tissue (102) by analysing the one or more images;
obtain one or more pseudo coloured images by false colouring the one or more images received;
determine image intensity ratio values of the one or more images captured by the HBM device (108) through the tailored filter (105c), transmitting light in wavelength range of 470-620 nm;
identify Regions of Interest (ROI) comprising a maximum change in the image intensity ratio value when compared to a standard ratio value obtained from normal/healthy tissues of similar anatomical sites; and
determine at least one grade of cancer or inflammation in the tissue (102) automatically based on the image intensity ratio of the oxygenated haemoglobin absorption and by comparing the image intensity ratio values obtained from the one or more images using an algorithm correlating the ratio values with pathological results of biopsy or inflammatory symptoms.

8. The system as claimed in claim 7, wherein the computing device (113) is further configured to superimpose at least one of the one or more images or the determined image intensity ratio values, to reduce false negatives during determination of the grade of cancer or inflammation in the tissue (102).

9. The system as claimed in claim 7, wherein the computing device (113) is connected with the Hand-held Biophotonic Medical (HBM) device (101) through a wired or wireless connection.

10. The system as claimed in claim 7, wherein the multimodal imaging includes but not limited to fluorescence, absorption and diffuse reflectance of tissues on illumination of tissues at 405, 545, 575 and 610 nm.

11. The system as claimed in claim 7, wherein the changes in intensity of oxygenated haemoglobin absorption are detected at 545, 575 and 610 nm in tissue (102).

12. The system as claimed in claim 7, wherein determination of at least one grade of cancer or inflammation in the tissue (102) is based on the image intensity ratios R545/R575, R610/R545 and R610/R575 of the oxygenated haemoglobin absorption at 545, 575 and 610 nm.

13. A method for multimodal and multispectral imaging of a tissue (102), comprising the steps of:
a) receiving, by a Hand-held Biophotonic Medical (HBM) device (101), one or more trigger pulses generated by a hardware switch (110) of the HBM device (101) when triggered manually or through a software trigger;
b) triggering, by the HBM device (101), one or more illumination devices (103a) of the HBM device (101), to illuminate the tissue (102) upon receiving the one or more trigger pulses, resulting in tissue fluorescence and/or diffuse reflectance of light upon absorption/scattering of an incident wavelength of light by the tissue (102);
c) controlling, by the HBM device (101), a miniature monochrome imaging device (108) of the HBM device (101), to capture one or more images of tissue fluorescence upon absorption of the incident light by constituents of the tissue (102) and/or to capture one or more images of diffusely reflected light due to multiple elastic scattering of the incident light at a predefined wavelength from the tissue (102) in real time using the miniature monochrome imaging device (108) and a collection optics unit (105) associated with the HBM device (101);
d) streaming, by the HBM device (101), a live video of tissue fluorescence wherein the live video is obtained using the miniature monochrome imaging device (108); and
e) transmitting, by the HBM device (101), the one or more images to a computing device (113) for processing of captured images and display of screening results;
wherein,
the multimodal imaging includes but is not limited to fluorescence, absorption, scattering and diffuse reflectance.

14. The method as claimed in claim 13, wherein said HBM device (109) is configured to: illuminate the tissue (102) with a predefined wavelength with predefined bandwidths resulting in tissue absorption, fluorescence, scattering and/or diffuse reflectance of light upon absorption of an incident wavelength by the tissue (102); capture one or more images of tissue absorption, fluorescence and diffuse reflectance in a predefined wavelength; and transmit the background image and one or more images of tissue absorption, fluorescence and diffuse reflectance to the computing device (113) for display in real time.

15. The method as claimed in claim 13, wherein said computing device (113) is configured to:
receive the one or more images transmitted by said HBM device (101) in real time;
detect changes in intensity of oxygenated haemoglobin absorption in the predefined wavelength range in the tissue (102) by analysing the one or more images;
obtain one or more pseudo coloured images by false colouring the one or more images received;
determine image intensity ratio values of the one or more images captured by the HBM device (108) in the predefined wavelength range;
identify Regions of Interest (ROI) comprising a maximum change in the image intensity ratio values when compared to a predefined standard ratio value; and
determine at least one grade of cancer or inflammation in the tissue (102) automatically based on the intensity of the oxygenated haemoglobin absorption and by correlating the image intensity ratio values obtained from the one or more images using an algorithm.

16. The method as claimed in claim 15, wherein the computing device (113) is further configured to superimpose at least one of the one or more images or the determined image intensity ratio values, to reduce false negatives associated with the determination of the grade of cancer or inflammation in the tissue (102).

17. The method as claimed in claim 13, wherein said method further comprises:
capturing a background image of a lesion under ambient light without illuminating the tissue, and subtracting said background image from the images of illuminated tissue.

18. The method as claimed in claim 13, wherein the method is applicable in diagnosing a grade of cancer and/or inflammation in a tissue of a human subject, said tissue being present in cavity such as an oral cavity, oesophagus, cervix, larynx, pharynx, GI tract, colon and alike.

19. Use of an HBM device (101) for diagnosing a grade of cancer and/or inflammation in a tissue of a human subject, said tissue being present in cavity such as an oral cavity, oesophagus cervix, larynx, pharynx, GI tract, colon and alike.

20. Use of an HBM device (101) including adaptation for use in endoscopes for imaging internal organs of a human body.
